(19) United States
(12) Patent Application Publication
(10) Pub. No.: US 2024/0342266 A1
(43) Pub. Date: Oct. 17, 2024

(54) METHOD FOR TREATING TUMOR WITH COMBINATION OF EXOGENOUS ANTIGEN AND THERAPEUTIC AGENT

(71) Applicant: GUANGZHOU ANJIE BIOMEDICAL TECHNOLOGY CO., LTD., Guangzhou (CN)

(72) Inventors: Chao Zhou, Guangzhou (CN); Hong An, Guangzhou (CN); Ling Zhou, Guangzhou (CN); Yongbiao Du, Guangzhou (CN); Pengyun Liao, Guangzhou (CN); Taoxi Wang, Guangzhou (CN); Haibin Michael Yin, Guangzhou (CN)

(21) Appl. No.: 18/755,746

(22) Filed: Jun. 27, 2024

Related U.S. Application Data
(63) Continuation of application No. PCT/CN2022/138691, filed on Dec. 13, 2022.

(30) Foreign Application Priority Data
Dec. 27, 2021 (CN) 202111617729.X

(51) Int. Cl. A61K 39/12; A61K 39/00; A61K 41/00; A61P 35/00; C12N 7/00; C12N 15/86

(52) U.S. Cl. CPC A61K 39/12; A61K 39/4611; A61K 39/4613; A61K 39/4615; A61K 39/4631; A61K 39/4632; A61K 39/464838; A61K 41/0047; A61P 35/00; C12N 7/00; C12N 15/86; A61K 2039/5256; A61K 2039/53; A61K 2039/585; C12N 2710/10032; C12N 2710/10043; C12N 2710/10051; C12N 2740/15043

(57) ABSTRACT

The present application belongs to the technical field of gene therapy, and discloses a method for treating a tumor with a combination of an exogenous antigen and a therapeutic agent. The present application also discloses a composition including an exogenous antigen and a therapeutically effective amount of a therapeutic agent. With the exogenous antigen as a target, the therapeutic agent kills a tissue or cell carrying the exogenous antigen and does not act on any tissue or cell without the exogenous antigen, thereby specifically killing the tissue or cell, such as a tumor cell. Since the exogenous antigen can be expressed in different types of tumors in different individuals, the method of the present application is a broad-spectrum anti-tumor method.

### CROSS-REFERENCE TO RELATED APPLICATIONS

The present application is a continuation application of PCT application No. PCT/CN2022/138691 filed on Dec. 13, 2022, which claims the benefit of Chinese Patent Application No. 202111617729.X filed on Dec. 27, 2021. The contents of all of the aforementioned applications are incorporated by reference herein in their entirety.

### REFERENCE TO SEQUENCE LISTING

This application includes a Sequence Listing filed electronically as a XML file named “Sequence listing_SCH-24045-USPT.xml”, created on Jun. 27, 2024, with a size of 19,592 bytes. The Sequence Listing is incorporated herein by reference.

### TECHNICAL FIELD

The present application belongs to the technical field of gene therapy, and specifically relates to a method for treating a tumor with a combination of an exogenous antigen and a therapeutic agent.

### BACKGROUND

Conventional therapeutic methods for cancer include surgery, radiotherapy, chemotherapy, monoclonal antibodies, and etc. In recent years, the cancer immunotherapy has attracted great attention because of its significant efficacy on advanced tumors unresponsive to conventional therapies. In 2013, the cancer immunotherapy was listed as the top ten scientific and technological breakthroughs of the year by Science. The cancer immunotherapy brings hope to patients with advanced tumors. In the cancer immunotherapy, the recognition and killing of an immune system to tumor cells are reactivated and maintained to restore a normal anti-tumor immune response in the body, thereby controlling and eliminating the tumor. The research on the cancer immunotherapy still faces many problems.

The tumor heterogeneity is a primary challenge to allow the accurate diagnosis and treatment and the cure of tumors. The tumor heterogeneity refers to large differences in terms of the genotype and the phenotype among different patient individuals with the same malignant tumor and among tumor cells at different parts of the same patient individual. The tumor heterogeneity is a common and crucial manifestation feature in the tumor evolution process, and plays an important role in the formation, development, and drug resistance of the tumor. The antigen heterogeneity is an important factor causing tumor recurrence. The chimeric antigen receptor T (CAR-T) cell therapy exhibits a significantly-improved therapeutic effect for B-cell leukemia and lymphoma, but the recurrence after the CAR-T cell therapy is still a problem. Up to 50% of patients who receive the CAR19-T cell therapy undergo recurrence within the first year after the CAR19-T cell therapy, and a significant proportion of these patients undergoing recurrence have a manifestation of CD19 antigen loss. To address the problem of antigen escape caused by tumor antigen heterogeneity, researchers have previously developed a tandem bispecific CAR20-19 construct targeting CD19 and CD20 antigens, which has exhibited promising efficacy and tolerability in phase I clinical trials. In order to solve the problem of tumor antigen heterogeneity and improve an anti-tumor effect of the body to glioblastoma multiforme (GBM), Marcela introduces BiTE against wild-type epidermal growth factor receptor (EGFR) (CART-BiTE) into CART-EGFRvIII. Although the development of multi-target drugs (such as dual CAR and multi-specific antibodies) can improve the therapeutic effects for antigenically heterogeneous tumors to some extent, it cannot solve the problem fundamentally. Under the pressure of drug selection, tumors expressing drug-targeted tumor antigens can be eliminated, but other tumor cells that do not express the targeted antigens are insensitive to drugs and can continue to grow. It is inevitably hard for the limited drugs to target tumor cells with complicated and diverse antigens.

The lack of specific targets is another challenge for the cancer immunotherapy. Targets for the current tumor therapeutic drugs are mainly tumor-associated antigens, which have potential off-target toxicity. Although some neoantigens caused by mutations have been found, these neoantigens are only expressed in a small number of tumor cells and drugs targeting these neoantigens are ineffective for most tumor cells that do not express these neoantigens. In addition, the discovery of neoantigens requires high-throughput sequencing, high individualization, and a high cost, which limits the development of drugs targeting neoantigens.

Therefore, solving the problems such as tumor antigen heterogeneity and lack of specific targets is the key to the development of safe and effective anti-tumor drugs.

### SUMMARY

In a first aspect, an objective of the present application is to provide a method for expressing an exogenous antigen in a tumor cell.

In a second aspect, an objective of the present application is to provide a composition.

In a third aspect, an objective of the present application is to provide an anti-tumor drug including the composition in the second aspect.

In a fourth aspect, an objective of the present application is to provide a method for treating a tumor.

To allow the above objectives, the present application adopts the following technical solutions:

In the first aspect of the present application, a method for expressing an exogenous antigen in a tumor cell is provided, including: introducing the exogenous antigen into the tumor cell, where the exogenous antigen is one selected from the group consisting of (1) and (2):
(1) a non-human protein or polypeptide, namely, a protein or polypeptide that is not expressed in a human body; and
(2) a nucleic acid encoding the non-human protein or polypeptide in (1).

Preferably, the nucleic acid includes DNA and RNA and further includes DNA and mRNA.

Preferably, the non-human protein or polypeptide includes, but is not limited to, a protein or polypeptide from bacteria, yeasts, protozoa or viruses, or a synthetic protein or polypeptide.

Further preferably, the non-human protein or polypeptide is an F protein of a respiratory syncytial virus (RSV), and has an amino acid sequence shown in SEQ ID NO: 2.

Further preferably, a nucleotide sequence of the nucleic acid encoding the non-human protein or polypeptide is shown in SEQ ID NO: 1.

Preferably, the exogenous antigen is transfected into a tumor cell through a delivery vector or electroporation.

Preferably, the delivery vector is a tumor-selective delivery vector.

Preferably, the tumor-selective delivery vector is selected from the group consisting of a natural polymer, a synthetic polymer, a cationic peptide, a cell-penetrating peptide, a biodegradable nanoparticle, a liposome, a lipid complex, a polymeric complex, a micelle, a dendritic polymer, a gel, a mucosal adhesive, a silicon nanoneedle, a gold nanoparticle, an exosome, a virus, and a pseudovirus.

Preferably, the virus includes a lentivirus, an adenovirus, and an adeno-associated virus.

Preferably, the virus is an oncolytic virus.

Preferably, the oncolytic virus is at least one selected from the group consisting of an adenovirus, a vaccinia virus, a Sindbis virus, a Seneca Valley virus, a coxsackievirus, a measles virus, a reovirus, a cowpox virus, a newcastle disease virus, a vesicular stomatitis virus, a herpes simplex virus, a poliovirus, an influenza virus, a mumps virus, and a parvovirus, and is further an adenovirus.

Preferably, the tumor includes lung cancer, liver cancer, breast cancer, gastric cancer, esophageal cancer, melanoma, head and neck cancer, prostate cancer, and pancreatic cancer.

Preferably, the method for expressing an exogenous antigen in a tumor cell includes infecting the tumor cell with an oncolytic adenovirus expressing an F protein of RSV as the exogenous antigen.

Preferably, a preparation method of the oncolytic adenovirus includes the following steps:
S1: inserting a target gene including a nucleotide sequence encoding the F protein of the RSV into a vector to obtain a target gene-containing vector, and cleaving the target gene-containing vector with a single enzyme to obtain a linearized target gene-containing vector;
S2: transforming the linearized target gene-containing vector and a pAdEasy-1 plasmid containing a type 5 adenovirus backbone into a competent cell to obtain a recombinant adenovirus vector, and cleaving the recombinant adenovirus vector with a single enzyme to obtain a linearized recombinant adenovirus vector; and
S3: transfecting the linearized recombinant adenovirus vector into a cell to obtain the oncolytic adenovirus.

Preferably, the target gene in the S1 further includes a human telomerase reverse transcriptase promoter (hTERTp), adenovirus E1A, and an internal ribosome entry site (IRES) sequence.

Preferably, a nucleotide sequence of the target gene in the S1 is shown in SEQ ID NO: 5.

Preferably, the vector in the S1 is a pShuttle vector.

Preferably, the target gene in the S1 is inserted between NotI and SalI cleavage sites of the vector.

Preferably, the single enzyme used in each of the S1 and the S2 is Pme I.

Preferably, the competent cell in the S2 is a competent cell BJ5183.

Preferably, the cell in the S3 is a trex293 cell.

Preferably, the tumor is prostate cancer or lung cancer.

Preferably, the method is a non-disease treatment method.

In the second aspect of the present application, a composition is provided, including an exogenous antigen and a therapeutically effective amount of a therapeutic agent,
where the exogenous antigen is one selected from the group consisting of (1) and (2):
(1) a non-human protein or polypeptide, namely, a protein or polypeptide that is not expressed in a human body; and
(2) a nucleic acid encoding the non-human protein or polypeptide in (1); and
with the exogenous antigen as a target, the therapeutic agent kills a tissue or cell carrying the exogenous antigen and does not act on any tissue or cell without the exogenous antigen, thereby specifically killing the tissue or cell carrying the exogenous antigen.

Preferably, the exogenous antigen and the therapeutic agent each exist independently in the composition without being mixed with each other.

Preferably, the tissue or cell is a tumor tissue or cell.

Preferably, the nucleic acid includes DNA and RNA and further includes DNA and mRNA.

Preferably, the non-human protein or polypeptide includes, but is not limited to, a protein or polypeptide from bacteria, yeasts, protozoa or viruses, or a synthetic protein or polypeptide.

Further preferably, the non-human protein or polypeptide is an F protein of RSV, and has an amino acid sequence shown in SEQ ID NO: 2.

Preferably, the exogenous antigen is transfected into the tissue or cell through a delivery vector or electroporation.

Preferably, the exogenous antigen is encapsulated in the delivery vector.

Preferably, the delivery vector is a tumor-selective delivery vector.

Preferably, the tumor-selective delivery vector is selected from the group consisting of a natural polymer, a synthetic polymer, a cationic peptide, a cell-penetrating peptide, a biodegradable nanoparticle, a liposome, a lipid complex, a polymeric complex, a micelle, a dendritic polymer, a gel, a mucosal adhesive, a silicon nanoneedle, a gold nanoparticle, an exosome, a virus, and a pseudovirus.

Preferably, the virus includes a lentivirus, an adenovirus, and an adeno-associated virus.

Preferably, the virus is an oncolytic virus.

Preferably, the oncolytic virus is at least one selected from the group consisting of an adenovirus, a vaccinia virus, a Sindbis virus, a Seneca Valley virus, a coxsackievirus, a measles virus, a reovirus, a cowpox virus, a newcastle disease virus, a vesicular stomatitis virus, a herpes simplex virus, a poliovirus, an influenza virus, a mumps virus, and a parvovirus, and is further an adenovirus.

Preferably, the tumor-selective delivery vector includes a tumor-targeted agent.

Preferably, the therapeutic agent includes a chimeric antigen receptor T cell (CAR-T cell), a T cell receptor modified T cell (TCR-T cell), a CAR-NK (natural killer) cell, an antigen-specific T cell, an antigen-specific DC (dendritic cell), a small-molecule targeted drug, and a monoclonal antibody; further, the therapeutic agent is a CAR-T cell and/or a monoclonal antibody; and furthermore, the therapeutic agent is a CAR-T cell.

Preferably, the CAR-T cell is obtained by introducing a chimeric antigen receptor (CAR) into a T lymphocyte.

Preferably, a method for introducing the CAR into the T lymphocyte includes lentivirus or retrovirus infection.

Further preferably, the CAR-T cell is obtained by infecting a T lymphocyte with a lentivirus carrying CAR.

Preferably, the CAR includes an antigen-binding domain targeting the exogenous antigen.

Preferably, the CAR further includes a transmembrane domain, a costimulatory domain, and an intracellular signaling domain.

Preferably, the exogenous antigen is an F protein of RSV, and has an amino acid sequence shown in SEQ ID NO: 2.

Preferably, an amino acid sequence of the CAR is shown in SEQ ID NO: 4.

Further preferably, a nucleotide sequence for the CAR is shown in SEQ ID NO: 3.

Preferably, the exogenous antigen is an oncolytic adenovirus expressing an F protein of RSV.

Preferably, a preparation method of the oncolytic adenovirus includes the following steps:
S1: inserting a target gene including a nucleotide sequence encoding the F protein of the RSV into a vector to obtain a target gene-containing vector, and cleaving the target gene-containing vector with a single enzyme to obtain a linearized target gene-containing vector;
S2: transforming the linearized target gene-containing vector and a pAdEasy-1 plasmid containing a type 5 adenovirus backbone into a competent cell to obtain a recombinant adenovirus vector, and cleaving the recombinant adenovirus vector with a single enzyme to obtain a linearized recombinant adenovirus vector; and
S3: transfecting the linearized recombinant adenovirus vector into a cell to obtain the oncolytic adenovirus.

Preferably, the target gene in the S1 further includes hTERTp, adenovirus E1A, and an IRES sequence.

Preferably, a nucleotide sequence of the target gene in the S1 is shown in SEQ ID NO: 5.

Preferably, the vector in the S1 is a pShuttle vector.

Preferably, the target gene in the S1 is inserted between NotI and SalI cleavage sites of the vector.

Preferably, the single enzyme used in each of the S1 and the S2 is Pme I.

Preferably, the competent cell in the S2 is a competent cell BJ5183.

Preferably, the cell in the S3 is a trex293 cell.

Preferably, the therapeutic agent is a CAR-T cell targeting an F protein of RSV.

Preferably, a preparation method of the CAR-T cell targeting an F protein of RSV includes the following steps:
S1: inserting CAR including an antigen-binding domain targeting the F protein of the RSV into a first lentiviral vector to obtain a second lentiviral vector in which the CAR including the antigen-binding domain targeting the F protein of the RSV is inserted;
S2: mixing the second lentiviral vector obtained in the S1 with a packaging plasmid to obtain a packaging system, transfecting the packaging system into an HEK 293T cell, and cultivating the HEK 293T cell to obtain a lentivirus; and
S3: infecting a T lymphocyte with the lentivirus to obtain the CAR-T cell targeting the F protein of the RSV.

Preferably, a nucleotide sequence for the CAR including the antigen-binding domain targeting the F protein of the RSV in the S1 is shown in SEQ ID NO: 3.

Preferably, the first lentiviral vector in the S1 is pRRLSIN.

Preferably, a preparation method of the pRRLSIN is as follows: replacing an ampicillin-resistant gene (ampR) of pRRLSIN.cPPT.PGK-GFP.WPRE with a kanamycin-resistant gene (KanR), and inserting a multiple cloning site between XhoI and SalI.

Preferably, in the S1, the CAR including the antigen-binding domain targeting the F protein of the RSV is inserted between BamHI and MluI of the first lentiviral vector pRRLSIN.

Preferably, the S1 further includes the following step: inserting an EF1α promoter and a c-Myc tag into the second lentiviral vector in which the CAR including the antigen-binding domain targeting the F protein of the RSV is inserted, which includes the following steps:
S11: amplifying an NheI-EF1α-Myc fragment with pLVX-EF1α-CAR 5E5 as a template and NheI-EF1α-F/SP-Myc-R as primers; and amplifying an Myc-Palivizumab-BB-MIuI fragment with pRRLSIN-Palivizumab-BB as a template and Myc-RSV CAR-F/CD3-MIuI-R as primers;
S12: amplifying EF1α-Myc-Palivizumab-BB with the NheI-EF1α-Myc fragment and the Myc-Palivizumab-BB-MIuI fragment as templates and NheI-EF1α-F/CD3-MIuI-R as primers; and
S13: cleaving the EF1α-Myc-Palivizumab-BB and the second lentiviral vector in which the CAR including the antigen-binding domain targeting the F protein of the RSV is inserted with NheI and MIuI, and ligating to obtain a third lentiviral vector.

Preferably, the packaging system in the S2 includes a second-generation three-plasmid packaging system and a third-generation four-plasmid packaging system.

Further preferably, the packaging system in the S2 is a third-generation four-plasmid packaging system, including: the second lentiviral vector obtained in the S1, pMDLg/pRRE, pRSV-REV, and pMD2.G.

Preferably, a method for the transfecting in the S2 includes, but is not limited to, electrotransfection, lipofection, polyethylenimine (PEI) transfection, or the like.

Preferably, a titer of the lentivirus in the S2 is (3−5)×10⁸ IU/mL.

Preferably, the T lymphocyte in the S3 is obtained by activating a peripheral blood mononuclear cell (PBMC), which is specifically as follows: cultivating the PBMC with a lymphocyte medium including an anti-human CD3 monoclonal antibody, a recombinant human interleukin 2, and plasma.

Preferably, the lymphocyte medium is a KBM 581 serum-free cell medium.

Preferably, the PBMC is isolated by a blood cell separator or Ficoll.

Preferably, the infecting a T lymphocyte with the lentivirus in the S3 is as follows: mixing the lentivirus, the T lymphocyte, and polybrene, and conducting centrifugal infection and cultivation.

In the third aspect of the present application, an anti-tumor drug including the composition in the second aspect is provided.

Preferably, the tumor includes lung cancer, melanoma, head and neck cancer, liver cancer, brain cancer, colorectal cancer, bladder cancer, breast cancer, ovarian cancer, uterine cancer, cervical cancer, lymphoma, gastric cancer, esophageal cancer, kidney cancer, prostate cancer, pancreatic cancer, and leukemia, and is further prostate cancer and/or lung cancer.

In the fourth aspect of the present application, a method for treating a tumor is provided, including: administering the exogenous antigen in the composition of the second aspect of the present application to a subject, whereby a tumor tissue or cell of the subject includes the exogenous antigen; and administering the therapeutic agent in the composition of the second aspect of the present application to the subject.

The present application has the following beneficial effects:

The present application provides a method for expressing an exogenous antigen in a tumor cell. With this method, the exogenous antigen can be introduced into tumor cells, whereby the tumor cells with antigen heterogeneity in a tumor tissue express the same specific antigen, which solves the problem of antigen heterogeneity faced by the cancer therapy. The exogenous antigen has high target specificity and strong immunogenicity, which solves the problem of lack of specific targets for the cancer therapy. With this method, the treatment of a tumor (an endogenous disease) is equivalent to the treatment of an infectious disease (there is a heterogenic antigen), which facilitates the development of safe and effective anti-tumor drugs.

The present application provides a composition including an exogenous antigen and a therapeutic agent. With the exogenous antigen as a target, the therapeutic agent kills a tissue or cell carrying the exogenous antigen and does not act on any tissue or cell without the exogenous antigen, thereby specifically killing the tissue or cell. That is, the composition can effectively kill tumor cells, which provides a new idea for the cancer therapy. In addition, the exogenous antigen can be expressed in different types of tumors in different individuals. Thus, the method of the present application is a broad-spectrum anti-tumor method and has great economic values and social significance.

### BRIEF DESCRIPTION OF THE DRAWINGS

FIG. 1 is a plasmid map of a shuttle vector pShuttle-hTERTp-E1A-IRES-RSV F in Example 1;

FIG. 2A and FIG. 2B show electrophoresis results of a recombinant adenovirus vector pAd-hTERTp-E1A-IRES-RSV F identified by Pac I cleavage in Example 1, where FIG. 2A shows electrophoresis results of plasmids of clones 1 to 10 after Pac I cleavage and FIG. 2B shows electrophoresis results of a plasmid of clone 8 after Pac I cleavage;

FIG. 3 shows electrophoresis results of a P2 recombinant oncolytic adenovirus OAd-hTERTp-E1A-IRES-RSV F identified by PCR in Example 1;

FIG. 4 shows flow cytometry assay results of the expression of an respiratory syncytial virus F protein (RSV-F) in trex293 cells infected with a P2 recombinant oncolytic adenovirus OAd-hTERTp-E1A-IRES-RSV F in Example 1;

FIG. 5 shows flow cytometry assay results of the expression of an RSV-F protein as an exogenous antigen in a prostate cancer cell line LnCap-FGC infected with an oncolytic adenovirus OAd-hTERTp-E1A-IRES-RSV F in Example 1;

FIG. 6 shows flow cytometry assay results of the expression of an RSV-F protein as an exogenous antigen in a non-small cell lung cancer cell line A549 infected with an oncolytic adenovirus OAd-hTERTp-E1A-IRES-RSV F in Example 1;

FIG. 7 shows western blotting assay results of the expression of an RSV-F protein as an exogenous antigen in a non-small cell lung cancer cell line A549 infected with an oncolytic adenovirus OAd-hTERTp-E1A-IRES-RSV F in Example 1;

FIG. 8 shows a change of a viral titer in a non-small cell lung cancer cell line A549 infected with an oncolytic adenovirus OAd-hTERTp-E1A-IRES-RSV F over time in Example 1;

FIG. 9 shows an oncolytic effect of an oncolytic adenovirus OAd-hTERTp-E1A-IRES-RSV F for a non-small cell lung cancer cell line A549 in Example 1;

FIG. 10 shows electrophoresis results of an NheI-EF1α-Myc fragment and an Myc-Palivizumab-BB-MIuI fragment amplified by PCR in Example 2;

FIG. 11 shows electrophoresis results of an EF1α-Myc-Palivizumab-BB fragment amplified by PCR in Example 2;

FIG. 12 shows electrophoresis results of a lentiviral expression vector pRRLSIN-EF1α-myc-Palivizumab-BB identified by colony PCR in Example 2;

FIG. 13 is a plasmid map of a lentiviral expression vector pRRLSIN-EF1α-myc-Palivizumab-BB in Example 2;

FIG. 14 shows flow cytometry assay results of positive rates of myc-Palivizumab-BB CAR-T cells in Example 2.

FIG. 15A and FIG. 15B show killing effects of combinations of a Palivizumab-BB CAR-T cell targeting RSV-F and an oncolytic adenovirus OAd-hTERTp-E1A-IRES-RSV F for lung cancer in Example 3, where FIG. 15A shows a killing effect of a combination of a Palivizumab-BB CAR-T cell targeting RSV-F and an oncolytic adenovirus OAd-hTERTp-E1A-IRES-RSV F (5TCID50) for lung cancer and FIG. 15B shows a killing effect of a combination of a Palivizumab-BB CAR-T cell targeting RSV-F and an oncolytic adenovirus OAd-hTERTp-E1A-IRES-RSV (10TCID50) for lung cancer; and

FIG. 16 shows test results of specificity of killing of a combination of a Palivizumab-BB CAR-T cell targeting RSV-F and an oncolytic adenovirus OAd-hTERTp-E1A-IRES-RSV F for a tumor in Example 4.

### DETAILED DESCRIPTION OF THE EMBODIMENTS

The content of the present application will be further described in detail below through specific examples.

All materials and reagents used in the following examples can be commercially-available materials and reagents, unless otherwise specified.

### Example 1 Expression of an Exogenous Antigen RSV-F in Tumor Cells

### 1. Construction of an Oncolytic Virus OAd-hTERTp-E1A-IRES-RSV F

(1) Construction of a Shuttle Vector pShuttle-hTERTp-E1A-IRES-RSV F

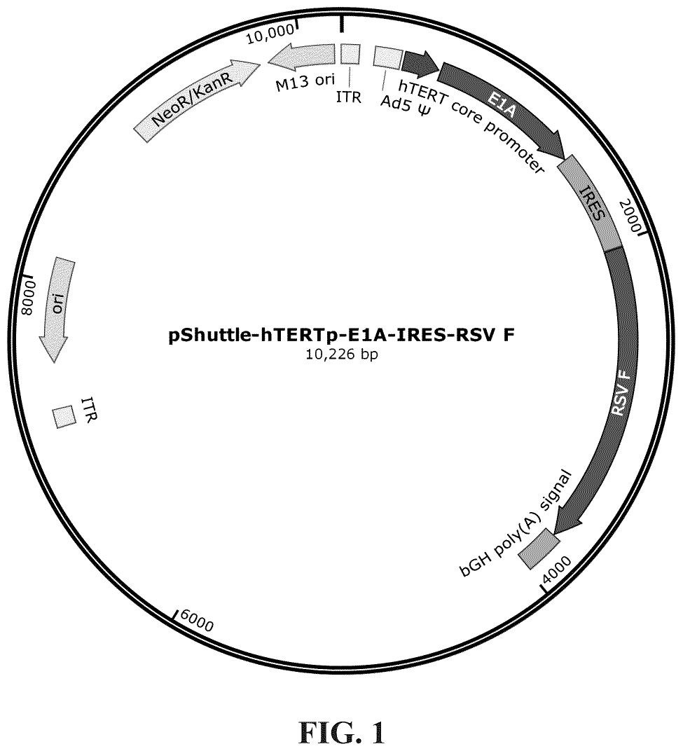

A pShuttle vector (purchased from Shanghai GeneBio Co., Ltd.) was adopted as a backbone to construct the shuttle vector, and a target fragment including hTERTp, adenovirus E1A, an IRES sequence, RSV-F (with a nucleotide sequence shown in SEQ ID NO: 1 and an amino acid sequence shown in SEQ ID NO: 2), and bGH poly (A) were inserted. In this example, the target fragment had a nucleotide sequence shown in SEQ ID NO: 5, and was synthesized by General Biologics and cloned between NotI and SalI cleavage sites of the pShuttle vector. The constructed shuttle vector was named pShuttle-hTERTp-E1A-IRES-RSV F, and a map of the shuttle vector was shown in FIG. 1. The plasmid was extracted, linearized through Pme I cleavage, and stored in a −20° C. freezer.

(2) Construction of a Recombinant Adenovirus Vector pAd-hTERTp-E1A-IRES-RSV F

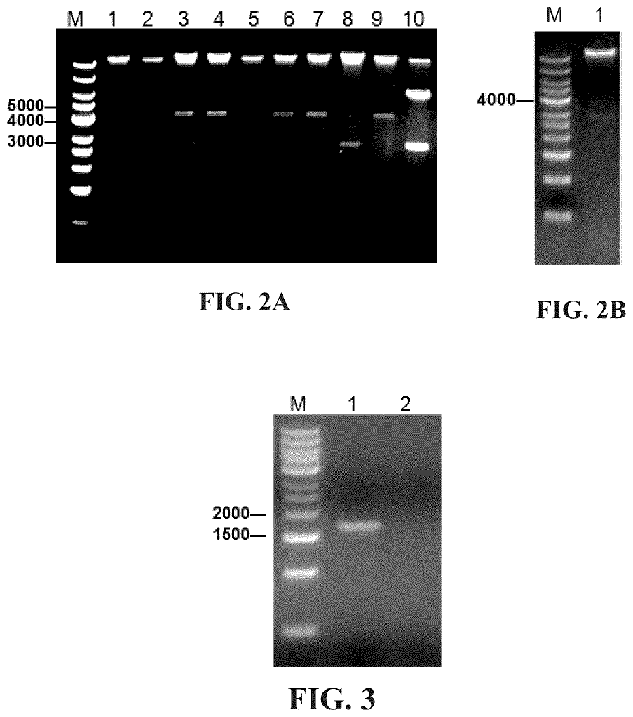

A linearized shuttle plasmid obtained in the step (1) and a pAdEasy-1 plasmid containing a type 5 adenovirus backbone (purchased from Shanghai GeneBio Co., Ltd.) were transformed into a competent cell BJ5183, and the recombinant adenovirus vector pAd-hTERTp-E1A-IRES-RSV F was constructed through homologous recombination. 10 single colonies were picked and inoculated into 3 mL of a medium and cultivated, and then plasmids were extracted and identified through Pac I cleavage. If the recombination was successful, a plasmid should be cleaved into 2 fragments, where one large fragment should be about 30 kb and the other small fragment should be 4.5 kb or 3 kb. As shown in FIG. 2A, plasmids of clones 3, 4, 6, 7, 8, and 9 each had 2 bands with correct sizes after the Pac I cleavage, indicating that these clones may all be positive clones. The clone 8 was arbitrarily selected, a plasmid of the clone 8 was transformed into a trans5α chemically competent cell (Beijing TransGen Biotech Co., Ltd.), then single colonies were picked and cultivated under shaking, and the plasmid was extracted and further identified through Pac I cleavage. As shown in FIG. 2B, 2 bands with correct sizes appeared, indicating that the recombinant adenovirus vector pAd-hTERTp-E1A-IRES-RSV F was successfully constructed. A resulting bacterial solution was subjected to expanded cultivation and cryopreserved.

### (3) Packaging of an Oncolytic Adenovirus OAd-hTERTp-E1A-IRES-RSV F

Trex293 cells were digested and prepared into a single-cell suspension, and the single-cell suspension was counted, inoculated into a 6-well plate at 1 ×10⁶ cells/well, and cultivated overnight in a 37° C. and 5% CO₂ incubator. The next day, a 1.5 mL EP tube was taken, 100 μL of a serum-free DMEM medium was added to the EP tube, then 20 μL of a plasmid solution obtained in the step (2) (PacI cleavage, an amount of a plasmid cleaved was about 1 μg) and 8 μL of a PEI 40K solution were added to the EP tube to obtain a mixed solution, and the mixed solution was gently pipetted up and down by a pipette tip for thorough mixing and then allowed to stand at room temperature for 20 min. During the co-incubation of a transfection reagent and a plasmid, a 6-well plate that had been coated one day in advance was taken out from a CO₂ incubator, a culture in each well was removed, a serum-free culture medium was added to the plate at 1 mL/well, and the plate was allowed to stand for 1 min; then a liquid in each well was discarded, then 0.9 mL of the serum-free medium was added to each well, and the plate was further incubated in a CO₂ incubator for 20 min; the plate was taken out from the incubator, a plasmid/PEI mixed solution was added to the plate, the plate was gently shaken in a shape of “∞” to allow thorough mixing and then incubated overnight in a CO₂ incubator, and the next day, the medium was replaced with a DMEM medium including 5% FBS; cells were continuously observed, and the medium change was conducted according to a cell status and a culture color until a cytopathic effect (CPE) appeared (this process usually took about 10 d); and the resulting cells and culture supernatant were directly collected, gently pipetted up and down for thorough mixing, repeatedly frozen and thawed 3 times at −80° C./room temperature, and centrifuged at 3,000 g for 10 min to obtain a supernatant, and the supernatant was collected to obtain a P1 virus solution.

### (4) Virus Amplification and Purification

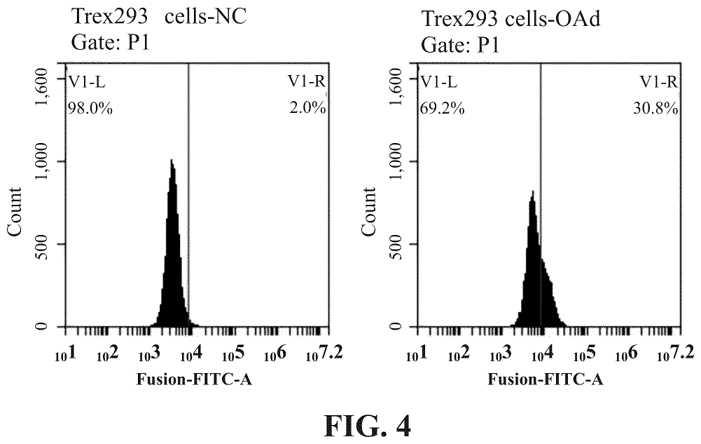

Trex293 cells were inoculated in 10 cm petri dishes and cultivated until a cell confluency reached 70% to 80%, and then an appropriate amount of the P1 virus solution was added to each petri dish. Cells were observed. When most of the cells were diseased after 2 d to 3 d of infection, a resulting culture was centrifuged to obtain a cell pellet, the cell pellet was collected, resuspended with a serum-free DMEM medium, repeatedly frozen and thawed 3 times, and centrifuged at 3,000 g for 10 min to obtain a supernatant, and the supernatant was collected to obtain a P2 virus solution. 100 μL of the P2 virus solution was taken and subjected to genomic DNA extraction, and the exogenous gene RSV F was amplified by PCR to identify the recombinant oncolytic adenovirus. A control group was set as follows: an equal proportion of trex293 cells were taken and repeatedly frozen and thawed to obtain a supernatant, and the supernatant was collected and subjected to genomic DNA extraction. Results were shown in FIG. 3, where lane1 was a band of PCR with genomic DNA extracted from the P2 virus solution as a template and lane2 was a band of PCR with genomic DNA extracted from the freeze-thaw supernatant of trex293 cells as a template. A band for the exogenous gene RSV F amplified with the genomic DNA extracted from the P2 virus solution as a template had a correct size (about 1,700 bp), indicating that an oncolytic adenovirus was successfully packaged in trex293 cells. Trex293 cells were infected with a P2 virus for 48 h, infected cells were collected, and the expression of RSV F was detected by flow cytometry. Trex293 cells not infected with the virus were adopted as a control. As shown in FIG. 4, the expression of RSV F was detected on a surface of OAd-infected trex293 cells, further indicating that the oncolytic adenovirus OAd-hTERTp-E1A-IRES-RSV F could be successfully packaged in trex293 cells and RSV F could be expressed normally in the cells. The P2 virus was further amplified in trex293 cells to obtain a P3 virus, the P3 virus was stored, and then a P4 virus was amplified with the P3 virus. The P1 to P4 viruses were used as virus seeds for the production of subsequent viruses.

Trex293 cells were subjected to expanded cultivation, inoculated into a 10-layer cell factory, and infected with the P4 virus. When most of the cells were diseased after 2 d to 3 d of the infection, a resulting culture was collected and centrifuged to obtain a cell culture supernatant and a cell pellet. The cell culture supernatant was temporarily stored in a 4° C. to 8° C. freezer. The cell pellet was repeatedly frozen and thawed 3 times and then centrifuged to obtain a supernatant, and the supernatant was collected and mixed with the cell culture supernatant to obtain an oncolytic virus stock solution. The oncolytic virus stock solution was purified through procedures such as clarification, a nuclease treatment, hollow fiber column concentration and buffer substitution, chromatography, hollow fiber column concentration, and sterilization filtration, and a purified oncolytic adenovirus OAd-hTERTp-E1A-IRES-RSV F solution was dispensed and stored in a −80° C. freezer. A tube of the purified virus solution was taken and tested for a viral titer by the Reed-Muench method, and the viral titer was 5.13×10⁹ TCID50/mL.

2. Infection of a Tumor Cell with the Oncolytic Adenovirus OAd-hTERTp-E1A-IRES-RSV F
(1) Detection of the Expression of the Exogenous Antigen in a Prostate Cancer Cell Line LnCap-FGC Infected with the Oncolytic Adenovirus

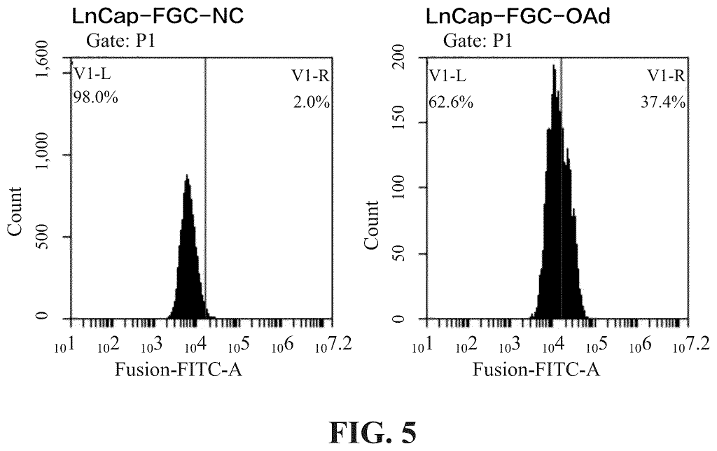

The prostate cancer cell line LnCap-FGC was inoculated into a 6-well cell culture plate, infected with the oncolytic virus at a multiplicity of infection (MOI) of 10TCID50, and tested by flow cytometry for the expression of RSV F on a cell surface, and LnCap-FGC cells not infected with the virus were adopted as a control. After 48 h of infection, a large number of cells floated, and the cells were collected and tested. Test results were shown in FIG. 5. As shown in this figure, the RSV F protein was expressed on a surface of prostate cancer cells infected with the oncolytic adenovirus, with a positive rate of about 35%.

(2) Expression of the Exogenous Antigen in a Non-Small Cell Lung Cancer Cell Line A549 Infected with the Oncolytic Adenovirus

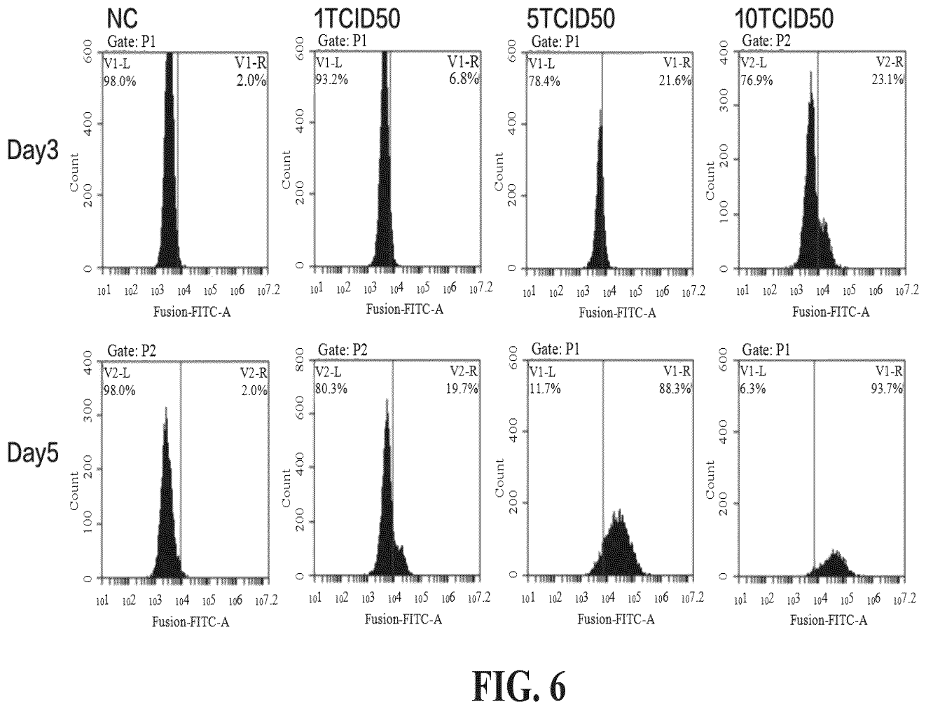

The non-small cell lung cancer cell line A549 was inoculated into a 24-well cell culture plate, infected with the oncolytic virus at MOIs of 1TCID50, 5TCID50, and 10TCID50, and tested at different time points by flow cytometry for the expression of RSV F on a cell surface, and A549 cells not infected with the virus were adopted as a control. Test results were shown in FIG. 6. As shown in this figure, after the tumor cell A549 was infected with OAd, the expression of the exogenous antigen RSV F on a surface of the tumor cell increased with the increase of a viral dose and an infection time. The positive rates at 5TCID50 and 10TCID50 reached about 20% on day 3 after oncolytic virus infection, and the positive rates at 5TCID50 and 10TCID50 significantly increased on day 5 after oncolytic virus infection, indicating that the exogenous antigen could be successfully expressed in lung cancer cells through the oncolytic virus. In particular, the RSV F protein was expressed on a surface of about 90% of A549 cells on day 5, whereby the tumor cells originally with antigen heterogeneity expressed the same antigen.

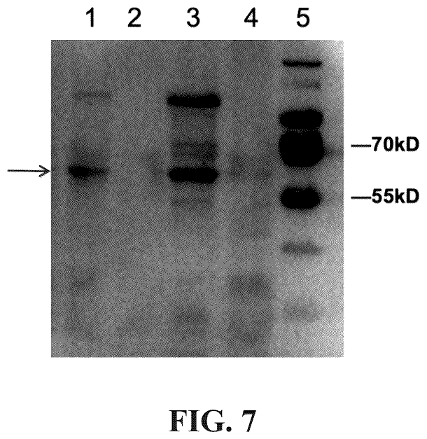

A549 cells and trex293 cells each were inoculated into a 6-well plate and infected with the oncolytic virus at MOI of 5TCID50 for 48 h, then cells were collected and subjected to total protein extraction, and the expression of RSV F was detected by western blotting. Detection results were shown in FIG. 7, where lane1 was for A549-OAd, lane2 was for A549-NC, lane3 was for trex293-OAd, lane4 was for trex293-NC, and lane5 was for PageRuler Prestained Protein Marker (THERMO). The expression of RSV F was detected in both A549 and trex293 cells infected with OAd, but the target band could not be detected in uninfected cells, which further indicated that the exogenous antigen could be expressed in cells infected with the oncolytic adenovirus OAd-hTERTp-E1A-IRES-RSV F.

### (3) Replication of the Oncolytic Adenovirus in the Lung Cancer Cell Line A549

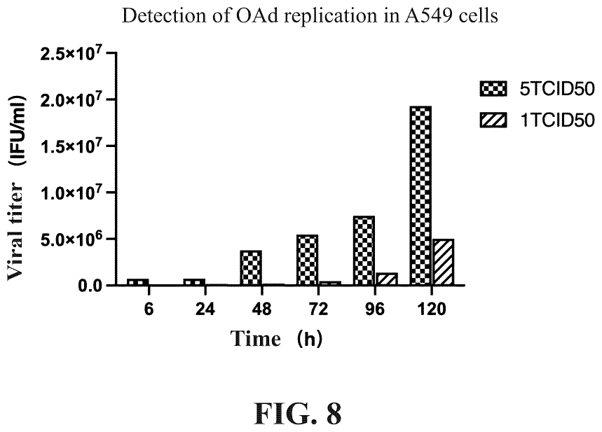

A549 cells were inoculated into a 6-well cell culture plate and infected with OAd-hTERTp-E1A-IRES-RSV F at MOIs of 1TCID50 and 5TCID50, and at different time points, a viral solution was collected and tested for a viral titer by Adeno-X™ Rapid Titer Kit (TAKARA). As shown in FIG. 8, the viral titer increased significantly over time, and at 5TCID50, a viral titer at 96 h (1.93×10⁷ IFU/mL) was 27 times a viral titer at 6 h (7.11×10⁵ IFU/mL), indicating that OAd could replicate in A549 cells.

(4) Oncolytic Effect of the Oncolytic Adenovirus for the Lung Cancer Cell Line A549

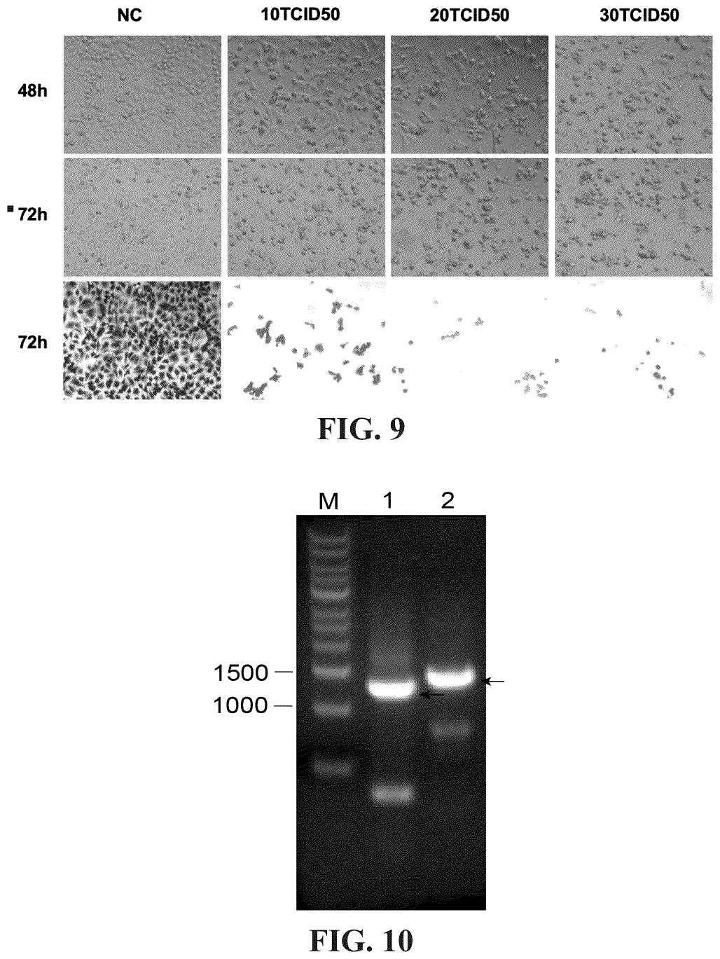

A549 cells were inoculated into a 6-well cell culture plate and infected with OAd-hTERTp-E1A-IRES-RSV F at MOIs of 10TCID50, 20TCID50, and 30TCID50. At 48 h and 72 h after infection, the growth of cells was observed under a microscope, and then the cells were stained with crystal violet. Results were shown in FIG. 9, where “48 h” showed images of cells at 48 h after virus infection under a microscope, “▪72 h” showed images of cells at 72 h after virus infection under a microscope, and “72 h” showed images of cells stained with crystal violet at 72 h after virus infection. In the control group, cells grew well, spread over the entire field of view, were spindle-shaped, and had intact membrane structures and excellent refractivity. In the oncolytic virus infection group, a cell density decreased, and cells shrunk and underwent cell membrane breakage. At the same time point, high-dose groups (20TCID50 and 30TCID50) had a lower cell density and a worse cell state than a low-dose group (10TCID50), and at the same dose, cells at 72 h had a lower cell density and a worse cell state than cells at 48 h, indicating that an oncolytic effect of the oncolytic adenovirus for the lung cancer cell line A549 was positively correlated to a dose and time. In addition, crystal violet staining results showed that a number of viable cells observed under a microscope in an oncolytic virus infection group was significantly reduced, and there were almost no viable cells in the high-dose group. The above results show that the oncolytic adenovirus OAd-hTERTp-E1A-IRES-RSV F has a significant killing effect for the lung cancer cell line A549.

### Example 2 Preparation of Palivizumab-BB CAR-T Cells Targeting RSV F as an Anti-Tumor Drug

1. Production of a Lentivirus pRRLSIN-EF1α-myc-Palivizumab-BB
(1) Construction of a Lentiviral Expression Vector pRRLSIN-EF1α-myc-Palivizumab-BB

A CAR structure was produced by linking an antigen-binding domain, a transmembrane domain, a costimulatory signaling domain, and an intracellular signaling domain in series, and a nucleotide sequence for CAR in this example was shown in SEQ ID NO: 3. The nucleotide sequence was synthesized artificially by General Biologics and cloned between BamHI and MluI cleavage sites of a lentiviral vector pRRLSIN (pRRLSIN was obtained by modifying pRRLSIN.cPPT.PGK-GFP.WPRE, which was specifically as follows: an ampicillin-resistant gene (AmpR) of pRRLSIN.cPPT.PGK-GFP.WPRE was replaced with a kanamycin-resistant gene (KanR) to obtain pRRLSIN.cPPT.PGK-GFP.WPRE (KanR), and a multiple cloning site was inserted between XhoI and SalI of pRRLSIN.cPPT.PGK-GFP.WPRE (KanR) to obtain pRRLSIN) to obtain a recombinant lentiviral expression vector named pRRLSIN-Palivizumab-BB. On this basis, an EF1α promoter was inserted in front of Palivizumab-BB, and in order to facilitate the detection of a positive rate of CAR-T cells subsequently, a c-Myc tag was inserted after a signal peptide sequence of the lentiviral expression plasmid Palivizumab-BB, so as to construct pRRLSIN-EF1α-Myc-Palivizumab-BB.

With pLVX-EF1α-CAR 5E5 (a specific construction method had been disclosed in the patent CN112940137A: PD-1 gene knockout MUC1-targeting CAR-T cell as well as preparation method and application of PD-1 gene knockout MUC1-targeting CAR-T cell) as a template and NheI-EF1α-F/SP-Myc-R as primers (a sequence of NheI-EF1α-F was as follows: CTAGCTAGCGCTCCGGTGCCCGTCAGT, SEQ ID NO: 6; and a sequence of SP-Myc-R was as follows: GAGGTCCTCTTCAGAGATAAGTTTTTGCTCCGGCCTGGCGGCGTGGA, SEQ ID NO: 7), an NheI-EF1α-Myc fragment was amplified; and with pRRLSIN-Palivizumab-BB as a template and Myc-RSV CAR-F/CD3-MIuI-R as primers (a sequence of Myc-RSV CAR-F was as follows: GAGCAAAAACTTATCTCTGAAGAGGACCTCCAAGTGACCCTGAGAGAGTCT, SEQ ID NO: 8; and a sequence of CD3-MIuI-R was as follows: CGACGCGTTTAGCGAGGGGGCAGGGCCT, SEQ ID NO: 9), an Myc-Palivizumab-BB-MIuI fragment was amplified. Results were shown in FIG. 10, where lane1 was for the NheI-EF1α-Myc fragment and lane2 was for the Myc-Palivizumab-BB-MIuI fragment.

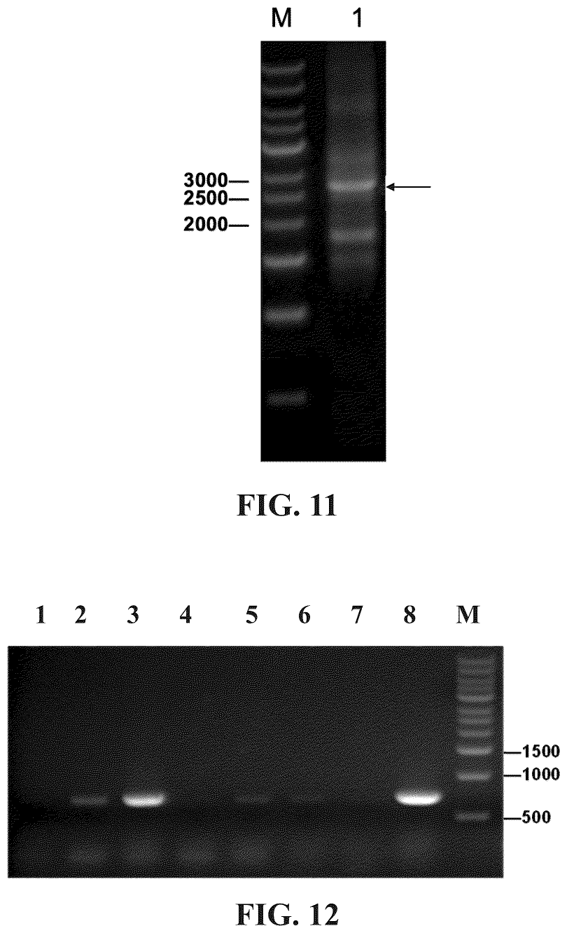

With products recovered from gels of lane1 and lane2 in FIG. 10 as a template (which was prepared according to the same molar concentration) and NheI-EF1α-F/CD3-MIuI-R as primers, EF1α-Myc-Palivizumab-BB was amplified by fusion PCR. Results were shown in FIG. 11.

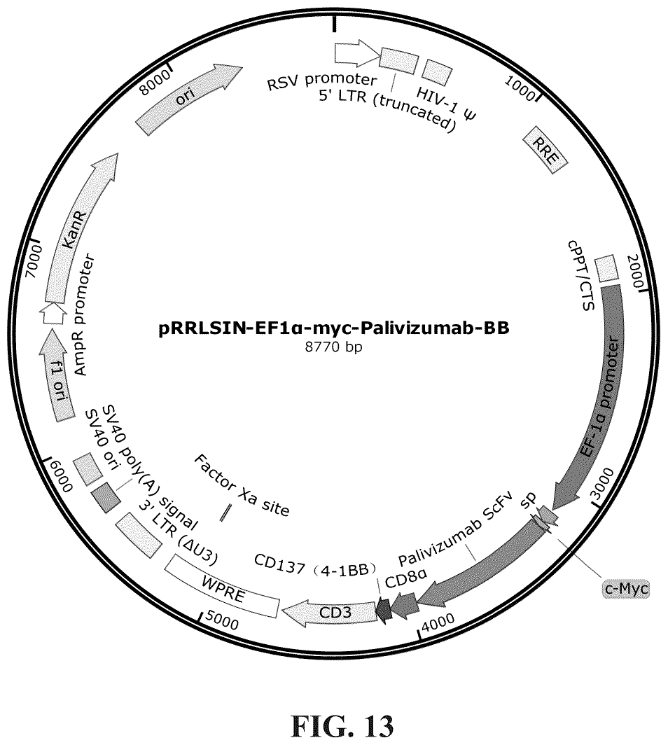

The target fragment EF1α-Myc-Palivizumab-BB and the vector pRRLSIN-Palivizumab-BB were cleaved by NheI and MIuI, cleavage products were recovered and ligated, and a ligation product was transformed into a trans5α chemically competent cell (Beijing TransGen Biotech Co., Ltd.). 8 single colonies were picked and identified by colony PCR. Results were shown in FIG. 12. An obvious target band was amplified for clones 3 and 8, indicating that the clones may be positive clones. The cell was cultivated under shaking, and then the plasmid was extracted and sequenced. A sequencing result indicated a correct sequence. The above results showed that the lentiviral expression vector pRRLSIN-EF1α-myc-Palivizumab-BB was successfully constructed. A plasmid map was shown in FIG. 13. The plasmid was extracted with an Endo-free Plasmid Maxi Kit (OMEGA) and stored in a =20° C. freezer.

(2) Packaging and Purification of the Lentivirus pRRLSIN-EF1α-myc-Palivizumab-BB

The cryopreserved HEK293T cells were thawed and sub-cultivated with a DMEM complete medium (DMEM medium+10% FBS). HEK293T cells were inoculated at a density of 3×10⁶ cells/mL into a 10-layer cell factory, 1 L of the DMEM complete medium was added, and the cells were cultivated overnight until a cell confluency reached 80% to 90% and then subjected to plasmid transfection. One T75 culture flask (flask A) was prepared, the lentiviral expression plasmid pRRLSIN-EF1α-myc-Palivizumab-BB, the lentiviral packaging plasmids pMDLg/pRRE (Kan⁺) and pRSV-REV (Kan+), and the lentiviral envelope plasmid pMD2.G (Kan³⁰) were added to the flask according to equal molar concentrations as final concentrations, and then serum-free DMEM was added to 60 mL. Another T75 flask (flask B) was prepared, 5.25 mL of 1 mg/mL PEI (polysciences) was added to the flask, and then serum-free DMEM was added to 60 mL. A solution A in the flask A and a solution B in the flask B each were thoroughly mixed and allowed to stand for 5 min. The solution B was added to the solution A to obtain a mixed solution, and the mixed solution was thoroughly mixed and allowed to stand for 20 min to obtain a DNA-PEI complex. The DNA-PEI complex was added to 1 L of a DMEM medium including 5% FBS to obtain a mixture, and the mixture was thoroughly mixed and used to replace a medium in the 10-layer cell factory. At 48 h after the transfection, about 1 L of a culture supernatant was collected and stored in a 2° C. to 8° C. freezer. 1 L of a fresh DMEM medium with 5% FBS was added to the 10-layer cell factory, and 24 h later, about 1 L of a culture supernatant was collected and stored in a 2° C. to 8° C. freezer. This process was repeated once. Approximately 3 L of culture supernatants collected 3 times were mixed and filtered through a capsule filter (SARTORIUS) for removing cells and cell debris to obtain a clear lentivirus supernatant. The clear lentivirus supernatant was concentrated by a Spectra/Por tangential flow filtration system (KROSFLO®KR2I) to 200 mL to 300 mL, filtered through a 0.45 μm filter membrane, and purified by chromatography to obtain a purified lentivirus solution. The purified lentivirus solution was subjected to sterilization filtration with a 0.22 μm filter (SARTORIUS), dispensed, and stored in a −80° C. freezer. A titer of the purified lentivirus was tested to be 3.41×10⁸ IU/mL.

2. Infection of a T Cell with the Lentivirus pRRLSIN-EF1α-myc-Palivizumab-BB to Prepare the CAR-T Cell

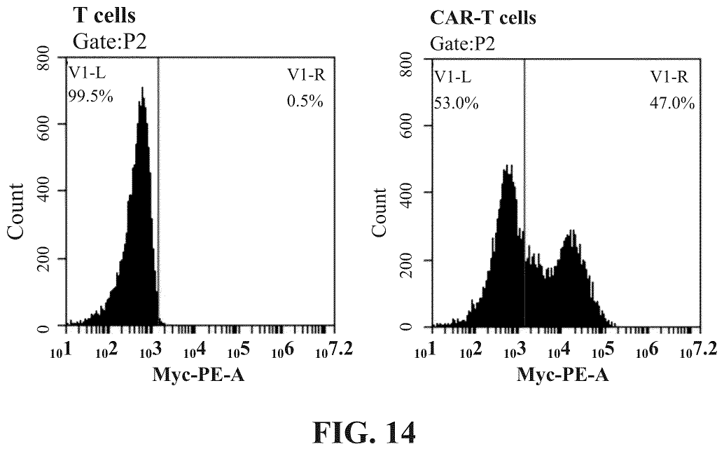

20 mL of peripheral blood of a healthy volunteer was collected, heparin was added to the peripheral blood for anticoagulation to obtain a mixture, the mixture was centrifuged to obtain serum and a cell pellet, and the serum was inactivated at 56° C. for later use. The cell pellet was diluted with normal saline, added to a centrifuge tube with a Ficoll solution, and subjected to density gradient centrifugation to obtain PBMCs, and the PBMCs were washed twice with normal saline and counted for later use. The PBMCs were resuspended with a KBM 581 Serum-free Cell Medium (CORNING) and adjusted to a cell density of 1-2×10⁶ cells/mL to obtain a PBMC suspension, the PBMC suspension was inoculated in a T75 cell culture flask, an anti-human CD3 monoclonal antibody (OKT-3) was added to the flask to activate the PBMCs, then 500 IU/mL of a recombinant human interleukin-2 (rhIL-2) and 5% to 10% of plasma were added to the flask, and the flask was incubated in a 37° C. and 5% CO² incubator. After the PBMCs were activated overnight, a resulting culture was counted and centrifuged to obtain a cell pellet, the cell pellet was resuspended with a KBM 581 Serum-free Cell Medium (recombinant human interleukin-2 (rhIL-2)) to a T lymphocyte density of 2-5×10⁶ cells/mL and evenly added to a 6-well plate, and the purified lentivirus solution (MOI=5) and polybrene (final concentration: 6 μg/mL) were added to the plate. Centrifugal infection was conducted at 700 g for 1.5 h, and then the plate was incubated in a 37° C. and 5% CO₂ incubator. 24 h after the lentivirus infection, a resulting culture was centrifuged to obtain a pellet, the pellet was resuspended in a KBM581 medium to obtain a suspension, 5% to 10% of plasma and 500 IU/mL of rhIL-2 were added to the suspension to obtain a mixture, and the mixture was incubated in a 37° C. and 5% CO₂ incubator. 72 h after the lentivirus infection, a positive rate of CAR-T cells was detected by flow cytometry. Results were shown in FIG. 14. A positive rate of myc-Palivizumab-BB CAR-T cells reached 47.0%.

### Example 3 Killing Effect of a Combination of a Palivizumab-BB CAR-T Cell Targeting RSV-F and an Oncolytic Adenovirus OAd-hTERTp-E1A-IRES-RSV F for Lung Cancer

### 1. Construction of a Target Cell A549-LUC

A non-small cell lung cancer cell line A549 was inoculated into a 6-well cell culture plate and cultivated in a 37° C. and 5% CO₂ incubator. The next day, the old medium in the plate was replaced with a fresh medium (RPMI-1640+10% FBS), the lentivirus pLVX-LUC-IRES-neo (the lentivirus carried a firefly luciferase and a neomycin-resistant gene neo and was stored in the company of the present disclosure, and a specific preparation method of the lentivirus was as follows: the firefly luciferase was inserted between XhoI and XbaI cleavage sites of a pLVX-IRES-Neo vector to obtain a lentiviral expression vector pLVX-LUC-IRES-neo, then pLVX-LUC-IRES-neo, a lentiviral packaging plasmid pSPAX2, and a lentiviral envelope plasmid pMD2.G were co-transfected into HEK293T cells at equal molar concentrations, and at 48 h and 72 h after the transfection, a cell culture supernatant was collected and centrifuged at 10,000 g for 5 min to obtain a supernatant which was the lentivirus pLVX-LUC-IRES-neo, and the supernatant was dispensed and stored in a −80° C. freezer) was added, and 4 h to 6 h later, the medium was changed and sub-cultivation was conducted. 72 h after the infection, G418 was added for screening. 5 d to 7 d later, monoclonal cells A549-LUC were screened by a limiting dilution method.

### 2. Luciferase-Based Cytotoxicity Test

(1) A549-LUC cells were digested to prepare a single-cell suspension as target cells. The cells were washed 2 times with an RPMI1640 medium and counted. The A549-LUC cells were divided into 2 parts, and the oncolytic adenovirus OAd-hTERTp-E1A-IRES-RSV F produced in Example 1 was added at MOIs of 10TCID50 and 5TCID50, respectively. The cells were resuspended at 1×10⁵ cells/mL (the virus and medium to be added were mixed first and then used to resuspend the cells).

(2) Two 96-well cell culture plates were taken, the two parts of cells were inoculated into the two 96-well cell culture plates at 100 μL/well (cell density: 1×10⁴ cells/well), respectively, and the cell culture plates were incubated in a 37° C. and 5% CO₂ incubator.

(3) After the oncolytic virus was incubated with the target cell for 48 h, the Palivizumab-BB CAR-T cell prepared in Example 2 and a T cell were taken out of an incubator as effector cells. Each culture was centrifuged at 500 g for 3 min to obtain a cell pellet, and the cell pellet was washed 2 times with an RPMI1640 medium, counted, and adjusted to a specified cell density. Each effector cell was added to the above 96-well plates according to effector cell/target cell ratios of 5, 10, and 20 to prepare experimental groups, namely, 0.5×10⁵ cells/50 μL/well, 1×10⁵ cells/50 μL/well, and 2×10⁵ cells/50 μL/well, respectively. A minimum lumen value group (MinCPS group) and a maximum lumen value group (MaxCPS group) for target cells were set, where the original medium in a MinCPS well was removed, an RPMI1640 medium including 1% Tween20 and 10% FBS was added at 150 μL/well to the MinCPS well, and an RPMI1640 medium including 10% FBS was added at 50 μL/well to the MaxCPS well. 3 replicate wells were set for each group. The culture plates were centrifuged at 300 g for 3 min and then further incubated in an incubator.

(4) The next day, the culture plates were taken out and centrifuged at 300 g for 4 min, and the medium in each well was removed according to 75 μL/well. A thawed SteadyGlo Reagent was thoroughly mixed and added to the culture plates at 75 μL/well. The culture plates were incubated in the dark for 5 min, each cell lysate supernatant was transferred at 100 μL/well to a detection blank plate, and a fluorescence intensity of each group was detected on a multi-mode microplate reader.

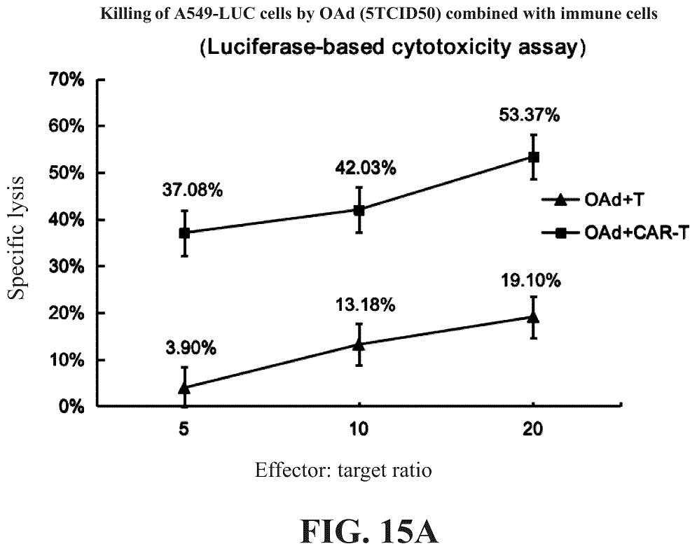

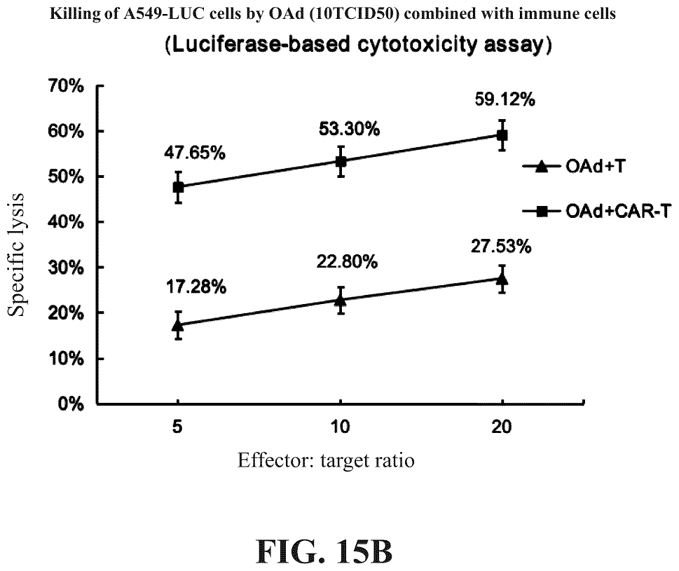

A killing efficiency was calculated according to the following formula: specific killing efficiency (%)=(MaxCPS−sample CPS)/(MaxCPS−MinCPS)×100%. Results were shown in FIG. 15A and FIG. 15B. A killing effect of a combination of an immune cell with an oncolytic adenovirus OAd-hTERTp-E1A-IRES-RSV F at 5TCID50 or 10TCID50 for A549-LUC increased with the increase of an effector cell/target cell ratio, and a combination of the CAR-T with the oncolytic virus is significantly better than a combination of the T cell with the oncolytic virus.

### Example 4 Specificity of Killing of a Combination of a Palivizumab-BB CAR-T Cell Targeting RSV-F and an Oncolytic Adenovirus OAd-hTERTp-E1A-IRES-RSV F for a Tumor

In order to demonstrate the specificity of the combination of the Palivizumab-BB CAR-T cell and the oncolytic adenovirus OAd-hTERTp-E1A-IRES-RSV F, Palivizumab-BB CAR-T (targeting an RSV F protein, CART RSV), CART 5E5 (targeting an MUC1 Tn antigen, A549 cells do not express MUC1 Tn and a preparation method of MUC1 Tn is referred to Example 2 in the patent document CN112940137A), and T cells were used in tests for killing A549-LUC cells and oncolytic adenovirus-infected A549-LUC cells.

(1) A549-LUC cells were digested to prepare a single-cell suspension as target cells according to Example 3. The cells were washed 2 times with an RPMI1640 medium and counted. The A549-LUC cells were divided into 2 parts, where the oncolytic adenovirus OAd-hTERTp-E1A-IRES-RSV F produced in Example 1 was added at MOI of 10TCID50 to one part of the cells, and no oncolytic virus was added to the other part of the cells. The two parts of cells each were resuspended at 1×10⁵ cells/mL. The two parts of cells were inoculated into 96-well cell culture plates at 100 μL/well (cell density: 1×10⁴ cells/well), and the cell culture plates were incubated in a 37° C. and 5% CO₂ incubator.

(2) 24 h later, the CART (RSV) and CART (5E5) prepared in Example 2 and T cells were taken out of an incubator as effector cells. Each culture was centrifuged at 500 g for 3 min to obtain a cell pellet, and the cell pellet was washed 2 times with an RPMI1640 medium, counted, and adjusted to a specified cell density. The effector cells each were added to the above 96-well plates at 1.5×10⁵ cells/50 μL/well as experimental groups. A MinCPS group and a MaxCPS group were set, where the original medium in a MinCPS well was removed, an RPMI1640 medium including 1% Tween20 and 10% FBS was added at 150 μL/well to the MinCPS well, and an RPMI1640 medium including 10% FBS was added at 50 μL/well to the MaxCPS well. 3 replicate wells were set for each group. The culture plates were centrifuged at 300 g for 3 min and then further incubated in an incubator.

(3) The next day, the culture plates were taken out and centrifuged at 300 g for 4 min, and the medium in each well was removed according to 75 μL/well. A thawed SteadyGlo Reagent was thoroughly mixed and added to the culture plates at 75 μL/well. The culture plates were incubated in the dark for 5 min, each cell lysate supernatant was transferred at 100 μL/well to a detection blank plate, and a fluorescence intensity of each group was detected on a multi-mode microplate reader.

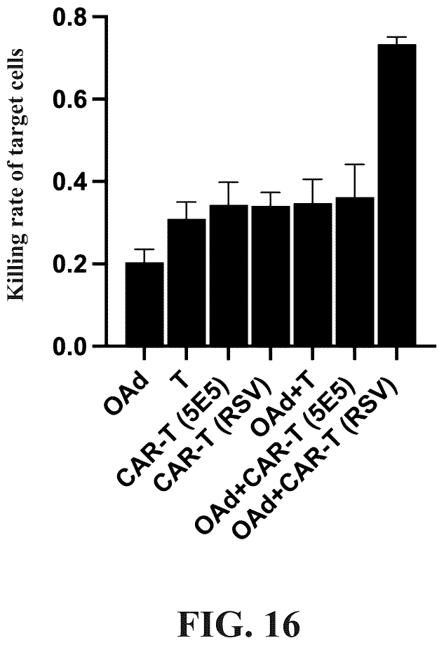

A killing efficiency was calculated according to the following formula: specific killing efficiency (%)=(MaxCPS−sample CPS)/(MaxCPS−MinCPS)×100%. Results were shown in FIG. 16. A killing effect of the CAR-T (RSV)+10TCID50 oncolytic adenovirus OAd-hTERTp-E1A-IRES-RSV F group for A549-LUC was significantly better than killing effects of the CAR-T (5E5)+10TCID50 oncolytic adenovirus group, the CAR-T (RSV) group, and the oncolytic adenovirus group, and there was no difference in terms of a killing effect for A549-LUC among the CAR-T (RSV) group, the CAR-T (5E5) group, and the T cell group, indicating that CAR-T (RSV) had no specific cytotoxicity for A549-LUC cells not expressing the exogenous antigen RSV F, but the oncolytic adenovirus OAd-hTERTp-E1A-IRES-RSV F could make A549-LUC cells express the exogenous antigen RSV F and thus specifically killed by CAR-T (RSV) targeting this antigen.

The above examples are preferred implementations of the present application. However, the implementations of the present application are not limited by the above examples. Any change, modification, substitution, combination, and simplification made without departing from the spiritual essence and principle of the present application should be an equivalent replacement manner, and all are included in the protection scope of the present application.

1. A composition, comprising an exogenous antigen and a therapeutically effective amount of a therapeutic agent,
wherein the exogenous antigen is one selected from the group consisting of (1) and (2):
(1) a non-human protein or polypeptide; and
(2) a nucleic acid encoding the non-human protein or polypeptide in (1); and
the therapeutic agent targets the exogenous antigen and acts on a tissue or cell comprising the exogenous antigen.

2. The composition according to claim 1, wherein
the non-human protein or polypeptide comprises a protein or polypeptide from bacteria, yeasts, protozoa or viruses, or a synthetic protein or polypeptide.

3. The composition according to claim 1, wherein the non-human protein or polypeptide is an F protein of a respiratory syncytial virus (RSV).

4. The composition according to claim 2, wherein
the exogenous antigen is transfected into the tissue or cell through a delivery vector or electroporation; and
the exogenous antigen is encapsulated in the delivery vector.

5. The composition according to claim 1, wherein
the therapeutic agent comprises a chimeric antigen receptor T cell (CAR-T cell), a T cell receptor modified T cell (TCR-T cell), a CAR-NK (natural killer) cell, an antigen-specific T cell, an antigen-specific DC (dendritic cell), a small-molecule targeted drug, and a monoclonal antibody.

6. The composition according to claim 5, wherein the therapeutic agent is the CAR-T cell and/or the monoclonal antibody.

7. The composition according to claim 6, wherein
a chimeric antigen receptor (CAR) of the CAR-T cell comprises an antigen-binding domain targeting the exogenous antigen.

8. The composition according to claim 7, wherein the CAR further comprises a transmembrane domain, a costimulatory domain, and an intracellular signaling domain.

9. The composition according to claim 7, wherein an amino acid sequence of the CAR is shown in SEQ ID NO: 4.

10. The composition according to claim 1, wherein
the composition comprises an oncolytic adenovirus expressing an F protein of RSV and a CAR-T cell targeting the F protein of the RSV.

11. The composition according to claim 10, wherein
a preparation method of the oncolytic adenovirus comprises the following steps:
S1: inserting a target gene comprising a nucleotide sequence encoding the F protein of the RSV into a vector to obtain a target gene-containing vector, and cleaving the target gene-containing vector with a single enzyme to obtain a linearized target gene-containing vector;
S2: transforming the linearized target gene-containing vector and a pAdEasy-1 plasmid containing a type 5 adenovirus backbone into a competent cell to obtain a recombinant adenovirus vector, and cleaving the recombinant adenovirus vector with a single enzyme to obtain a linearized recombinant adenovirus vector; and
S3: transfecting the linearized recombinant adenovirus vector into a cell to obtain the oncolytic adenovirus.

12. The composition according to claim 10, wherein a preparation method of the CAR-T cell targeting the F protein of the RSV comprises the following steps:
S1: inserting a CAR comprising an antigen-binding domain targeting the F protein of the RSV into a first lentiviral vector to obtain a second lentiviral vector in which the CAR comprising the antigen-binding domain targeting the F protein of the RSV is inserted;
S2: mixing the second lentiviral vector obtained in the S1 with a packaging plasmid to obtain a packaging system, transfecting the packaging system into an HEK 293T cell, and cultivating the HEK 293T cell to obtain a lentivirus; and
S3: infecting a T lymphocyte with the lentivirus to obtain the CAR-T cell targeting the F protein of the RSV.

13. An anti-tumor drug, comprising the composition according to claim 1.

14. An anti-tumor drug, comprising the composition according to claim 10.

15. The anti-tumor drug according to claim 13, wherein a tumor targeted by the anti-tumor drug comprises lung cancer, melanoma, head and neck cancer, liver cancer, brain cancer, colorectal cancer, bladder cancer, breast cancer, ovarian cancer, uterine cancer, cervical cancer, lymphoma, gastric cancer, esophageal cancer, kidney cancer, prostate cancer, pancreatic cancer, and leukemia.

16. The anti-tumor drug according to claim 14, wherein a tumor targeted by the anti-tumor drug comprises lung cancer, melanoma, head and neck cancer, liver cancer, brain cancer, colorectal cancer, bladder cancer, breast cancer, ovarian cancer, uterine cancer, cervical cancer, lymphoma, gastric cancer, esophageal cancer, kidney cancer, prostate cancer, pancreatic cancer, and leukemia.

17. A method for treating a tumor, comprising:
administering the exogenous antigen in the composition according to claim 1 to a subject, whereby a tumor tissue or cell in the subject comprises the exogenous antigen; and
administering the therapeutic agent in the composition according to claim 1 to the subject.

18. The method according to claim 17, wherein the tumor comprises lung cancer, melanoma, head and neck cancer, liver cancer, brain cancer, colorectal cancer, bladder cancer, breast cancer, ovarian cancer, uterine cancer, cervical cancer, lymphoma, gastric cancer, esophageal cancer, kidney cancer, prostate cancer, pancreatic cancer, and leukemia.

19. A method for treating a tumor, comprising:
administering the exogenous antigen in the composition according to claim 10 to a subject, whereby a tumor tissue or cell in the subject comprises the exogenous antigen; and
administering the therapeutic agent in the composition according to claim 10 to the subject.

20. The method according to claim 19, wherein the tumor comprises lung cancer, melanoma, head and neck cancer, liver cancer, brain cancer, colorectal cancer, bladder cancer, breast cancer, ovarian cancer, uterine cancer, cervical cancer, lymphoma, gastric cancer, esophageal cancer, kidney cancer, prostate cancer, pancreatic cancer, and leukemia.


SEQUENCE LISTING (9 sequences)

SEQ ID NO: 1 - DNA, 1725 bp, Human orthopneumovirus
  source 1..1725: mol_type = genomic DNA
atggagttgccaatcctcaaaacaaatgcaattaccgcaatccttgctgcagtcacactctgttttgcttccagtcaaaacatcactgaagaattttatcaaacaacatgcagtgcagtcagcaaaggctatcttagtgctctaagaactggttggtatactagtgttataactatagaattaagtaatatcaaggaaaataagtgtaatggaacagacgctaaggtaaaattgataaaacaagaattagataaatataaaagtgctgtaacagaattgcagttgctcatgcaaagcacaccggcaaccaacaatcgagccagaagagaactaccaaggtttatgaattatacactcaacaataccaaaaataccaatgtaacattaagcaagaaaaggaaaagaagatttcttggctttttgttaggtgttggatctgcaatcgccagtggcattgctgtatctaaggtcctgcacctagaaggggaagtgaacaaaatcaaaagtgctctactatccacaaacaaggctgtagtcagcttatcaaatggagttagtgtcttaaccagcaaagtgttagacctcaaaaactatatagataaacagttgttacctattgtgaacaagcaaagctgtagcatatcaaacattgaaactgtgatagagttccaacaaaagaacaacagactactagagattaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagttactctatcatgtccataataaaggaggaagtcttagcatatgtagtacaattaccactatatggtgtaatagatacaccttgttggaaactgcacacatcccctctatgtacaaccaacacaaaggaagggtccaacatctgtttaacaagaaccgacagaggatggtactgtgacaatgcaggatcagtatctttcttcccactagctgaaacatgtaaagttcaatcgaatcgagtattttgtgacacaatgaacagtttaacattaccaagtgaagtaaatctctgcaacattgacatattcaaccccaaatatgattgcaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataagggggttgacactgtgtctgtaggtaatacattatattatgtaaataagcaagaaggcaaaagtctctatgtaaaaggtgaaccaataataaatttctatgacccattagtgttcccctctgatgaatttgatgcatcaatatctcaagtcaatgagaagattaaccagagcctagcatttattcgtaaatccgatgaattattacataatgtaaatgctggtaaatccaccataaatatcatgataactactataattatagtgattatagtaatattgttatcattaattgccgttggactgctcctatactgcaaggccagaagcacaccagtcacactaagcaaggatcaactgagtggtataaataatattgcatttagtaactaa

SEQ ID NO: 2 - protein, 574 aa, Human orthopneumovirus
MELPILKTNAITAILAAVTLCFASSQNITEEFYQTTCSAVSKGYLSALRTGWYTSVITIELSNIKENKCNGTDAKVKLIKQELDKYKSAVTELQLLMQSTPATNNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 3 - DNA, 1467 bp, synthetic construct
  source 1..1467: mol_type = other DNA
  misc_feature 1..1467: note = synthetic
atggccttaccagtgaccgccttgctcctgccgctggccttgctgctccacgccgccaggccgcaagtgaccctgagagagtctggacctgctctggtcaagcccacacagaccctgacactgacctgcaccttcagcggctttagcctgagcacaagcggcatgagcgtcggctggattagacagcctcctggcaaagccctggaatggctggccgacatttggtgggacgacaagaaggactacaaccccagcctgaagtcccggctgaccatcagcaaggacaccagcaagaaccaggtggtgctgaaagtgaccaacatggaccctgccgacaccgccacctactactgtgccagatccatgatcaccaactggtacttcgacgtgtggggagccggcaccacagtgaccgtctcttcaggcggcggcggatctggaggaggaggaagcggcggcggcggttctgacatccagatgacacagagccctagcacactgtctgccagcgtgggcgacagagtgaccatcacatgcaagtgccagctgagcgtgggctacatgcactggtatcagcaaaagcccggcaaggcccctaagctgctgatctacgatacctccaagctggcctctggcgtgccctccagattttctggcagcggcagcggaaccgagttcaccctgaccatctcaagcctgcagcctgacgacttcgctacgtactactgcttccaaggcagcggctaccccttcacatttggcggcggaacaaagctggaaatcaagcggaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgctcgagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - protein, 488 aa, synthetic construct
  REGION 1..488: note = synthetic
MALPVTALLLPLALLLHAARPQVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSSGGGGSGGGGSGGGGSDIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELLERVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 3630 bp, synthetic construct
  source 1..3630: mol_type = other DNA
  misc_feature 1..3630: note = synthetic
ccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctggtaccgccaccatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggatcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaacgcgtatggagttgccaatcctcaaaacaaatgcaattaccgcaatccttgctgcagtcacactctgttttgcttccagtcaaaacatcactgaagaattttatcaaacaacatgcagtgcagtcagcaaaggctatcttagtgctctaagaactggttggtatactagtgttataactatagaattaagtaatatcaaggaaaataagtgtaatggaacagacgctaaggtaaaattgataaaacaagaattagataaatataaaagtgctgtaacagaattgcagttgctcatgcaaagcacaccggcaaccaacaatcgagccagaagagaactaccaaggtttatgaattatacactcaacaataccaaaaataccaatgtaacattaagcaagaaaaggaaaagaagatttcttggctttttgttaggtgttggatctgcaatcgccagtggcattgctgtatctaaggtcctgcacctagaaggggaagtgaacaaaatcaaaagtgctctactatccacaaacaaggctgtagtcagcttatcaaatggagttagtgtcttaaccagcaaagtgttagacctcaaaaactatatagataaacagttgttacctattgtgaacaagcaaagctgtagcatatcaaacattgaaactgtgatagagttccaacaaaagaacaacagactactagagattaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagttactctatcatgtccataataaaggaggaagtcttagcatatgtagtacaattaccactatatggtgtaatagatacaccttgttggaaactgcacacatcccctctatgtacaaccaacacaaaggaagggtccaacatctgtttaacaagaaccgacagaggatggtactgtgacaatgcaggatcagtatctttcttcccactagctgaaacatgtaaagttcaatcgaatcgagtattttgtgacacaatgaacagtttaacattaccaagtgaagtaaatctctgcaacattgacatattcaaccccaaatatgattgcaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataagggggttgacactgtgtctgtaggtaatacattatattatgtaaataagcaagaaggcaaaagtctctatgtaaaaggtgaaccaataataaatttctatgacccattagtgttcccctctgatgaatttgatgcatcaatatctcaagtcaatgagaagattaaccagagcctagcatttattcgtaaatccgatgaattattacataatgtaaatgctggtaaatccaccataaatatcatgataactactataattatagtgattatagtaatattgttatcattaattgccgttggactgctcctatactgcaaggccagaagcacaccagtcacactaagcaaggatcaactgagtggtataaataatattgcatttagtaactaatctagactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 6 - DNA, 27 bp, synthetic construct
  source 1..27: mol_type = other DNA
  misc_feature 1..27: note = synthetic
ctagctagcgctccggtgcccgtcagt

SEQ ID NO: 7 - DNA, 47 bp, synthetic construct
  source 1..47: mol_type = other DNA
  misc_feature 1..47: note = synthetic
gaggtcctcttcagagataagtttttgctccggcctggcggcgtgga

SEQ ID NO: 8 - DNA, 51 bp, synthetic construct
  source 1..51: mol_type = other DNA
  misc_feature 1..51: note = synthetic
gagcaaaaacttatctctgaagaggacctccaagtgaccctgagagagtct

SEQ ID NO: 9 - DNA, 28 bp, synthetic construct
  source 1..28: mol_type = other DNA
  misc_feature 1..28: note = synthetic
cgacgcgtttagcgagggggcagggcct